(19) United States
(12) Patent Application Publication
(10) Pub. No.: US 2011/0196154 A1
(43) Pub. Date: Aug. 11, 2011

(54) AMORPHOUS PEMETREXED DISODIUM

(75) Inventors: Rajasekhar Kadaboina, Hyderabad (IN); Sekhar Munaswamy Nariyam, Hyderabad (IN); Srividya Ramakrishnan, Hyderabad (IN); Subbareddy Peddireddy, Kadapa (IN); Mohammed Azeezulla Baig, Vijayawada (IN); Nagakiran Duggirala, Krishna (IN)

(73) Assignees: Dr. Reddy's Laboratories Ltd., Hyderabad (IN); Dr. Reddy's Laboratories, Inc., Bridgewater, NJ (US)

(21) Appl. No.: 13/042,867

(22) Filed: Mar. 8, 2011

Related U.S. Application Data
(63) Continuation of application No. PCT/US2009/055815, filed on Sep. 3, 2009.
(60) Provisional application No. 61/120,946, filed on Dec. 9, 2008.
(60) Provisional application No. 61/153,039, filed on Feb. 17, 2009.
(60) Provisional application No. 61/218,677, filed on Jun. 19, 2009.

(30) Foreign Application Priority Data
Sep. 8, 2008 (IN) 2179/CHE/2008
Dec. 3, 2008 (IN) 3054/CHE/2008
Apr. 2, 2009 (IN) 773/CHE/2009

(51) Int. Cl. C07D 487/04

(52) U.S. Cl. 544/280

(57) ABSTRACT

Processes for the preparation of amorphous pemetrexed disodium, and removal of residual solvents from amorphous pemetrexed disodium.

### INTRODUCTION

Aspects of the present application relate to amorphous pemetrexed disodium and processes for its preparation.

The drug compound having the adopted name “pemetrexed disodium” has chemical names: N-[4-[2-(2-amino-4,7-dihydro-4-oxo-1H-pyrrolo[2,3-d]pyrimidin-5-yl)ethyl]benzoyl]L-glutamic acid disodium salt, heptahydrate; or 2-[4-[2-(4-amino-2-oxo-3,5,7-triazabicyclo[4.3.0]nona-3,8,10-trien-9-yl)ethyl]benzoyl]amino pentane dioic acid; and is represented by the structure of Formula I.

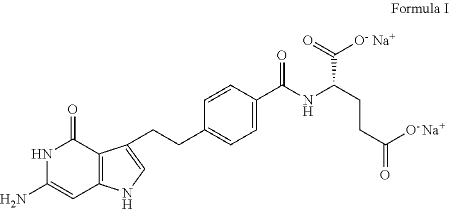

Pemetrexed is an anti-folate anti-neoplastic agent that exerts its action by disrupting folate-dependent metabolic processes essential for cell replication. It is believed to work by inhibiting three enzymes that are required in purine and pyrimidine biosynthesis-thymidylate synthase (TS), dihydrofolate reductase (DHFR), and glycinamide ribonucleotide formyl transferase (GARFT). Pemetrexed is available in the market under the brand name ALIMTA®.

Chelius et al., in International Application Publication No. WO 01/14379 A2, disclose pemetrexed disodium crystalline hydrate Form I and a process for preparation thereof. Chinese Patent Application 1778802 assigned to Chongging Pharmaceutical Research Institute discloses crystalline pemetrexed disodium trihydrate. Chelius et al., in U.S. Pat. No. 7,138,521, disclose pemetrexed disodium heptahydrate and a process for the preparation thereof. The process involves:

a) crystallizing pemetrexed disodium from a solution comprising pemetrexed disodium, water, and a water miscible solvent; and

b) drying the crystalline pemetrexed disodium with humid nitrogen.

Busolli et al., in International Application Publication No. WO 2008/021411 disclose a process for preparation of a lyophilized pharmaceutically acceptable salt of pemetrexed diacid, by reacting pemetrexed diacid or its salt with an agent capable of forming salt in a solvent suitable for lyophilization, wherein the pharmaceutically acceptable salt of pemetrexed diacid is not isolated prior to the lyophilization process. Busolli et al., in International Application Publication No. WO 2008/021405 A1, disclose seven crystalline forms of pemetrexed diacid designated as Forms A, B, C, D, E, F, and G, and processes for preparation thereof.

International Application Publication No. WO 2008/124485 discloses an amorphous form of the disodium salt of pemetrexed and a process for preparation thereof, the process comprising isolation of solid amorphous salt from a solution of pemetrexed disodium by spray drying or by freeze drying processes. Further, it discloses a polymorphic form designated as Form III, which is obtained from azeotropic distillation. Patel et al., in U.S. Patent Application Publication No. 2009/0181990, discloses an amorphous form of pemetrexed disodium and process for preparation thereof, the process comprising isolation of amorphous pemetrexed disodium from a solution of pemetrexed disodium by spray drying, freeze drying, flash drying, or any combination thereof.

There remains a continuing need to develop processes for preparing amorphous pemetrexed disodium, which are amenable to scale-up for larger production quantities and yield both formulation and therapeutic benefits.

### SUMMARY

An aspect of the present application provides processes for preparing an amorphous form of pemetrexed disodium.

In an embodiment, there is provided a process for preparing substantially pure amorphous pemetrexed disodium, comprising:
a) providing a solution of pemetrexed disodium in a solvent;
b) precipitating a solid from the solution of a) by combining with an anti-solvent; and
c) recovering solid amorphous pemetrexed disodium from b).

In an embodiment, there is provided a process for preparing pure amorphous pemetrexed disodium, comprising:
(a) providing a solution of pemetrexed disodium in an alcohol solvent;
(b) heating the solution obtained from (a) to about 55-65° C.; and
(c) recovering solid amorphous pemetrexed disodium from (b).

An aspect of the invention provides a process for preparing amorphous pemetrexed disodium, comprising:
(a) providing a solution of pemetrexed disodium in methanol below 35° C.;
(b) heating the solution to about 55-65° C.;
(c) cooling the solution; and
(d) recovering amorphous pemetrexed disodium.

An aspect of the invention provides a process for preparing amorphous pemetrexed disodium, comprising:
(a) providing a solution of pemetrexed disodium in methanol below 35° C.;
(b) heating the solution to about 55-65° C.;
(c) combining the solution with an anti-solvent; and
(d) recovering amorphous pemetrexed disodium.

An aspect of the invention provides a process for removing residual solvents from amorphous pemetrexed disodium, comprising:
(a) exposing amorphous pemetrexed disodium to relative humidity below about 75 percent at temperatures about 20° C. to about 40° C., for a time that reduces the residual solvent content; and
(b) optionally, drying exposed amorphous pemetrexed disodium at temperatures about 20° C. to about 50° C.

An aspect of the invention provides a process for preparing crystalline pemetrexed disodium heptahydrate, comprising:
(a) providing a solution of pemetrexed disodium in water with inert gas bubbling;
(b) precipitating the solid from the solution of (a) by combining with an anti-solvent with inert gas bubbling;
(c) isolating the solid from (b) under an inert gas atmosphere; and
(d) drying the solid of (c) under vacuum to afford crystalline pemetrexed disodium heptahydrate.

Pemetrexed disodium amorphous material of the present application is sufficiently stable and well suited for use in producing pharmaceutical formulations, which are useful in the treatment of disease, including, but are not limited to, non-small cell lung cancer.

### BRIEF DESCRIPTION OF THE DRAWINGS

FIG. 1 is an illustration of an X-ray powder diffraction pattern of amorphous pemetrexed disodium, prepared according to Example 3.

FIG. 2 is an illustration of a thermogravimetric analysis (TGA) curve of amorphous pemetrexed disodium, prepared according to Example 3.

FIG. 3 is an illustration of a Fourier-transform infrared (FT-IR) absorption spectrum of amorphous pemetrexed disodium, prepared according to Example 3.

FIG. 4 is an illustration of an X-ray powder diffraction pattern of amorphous pemetrexed disodium, prepared according to Example 5.

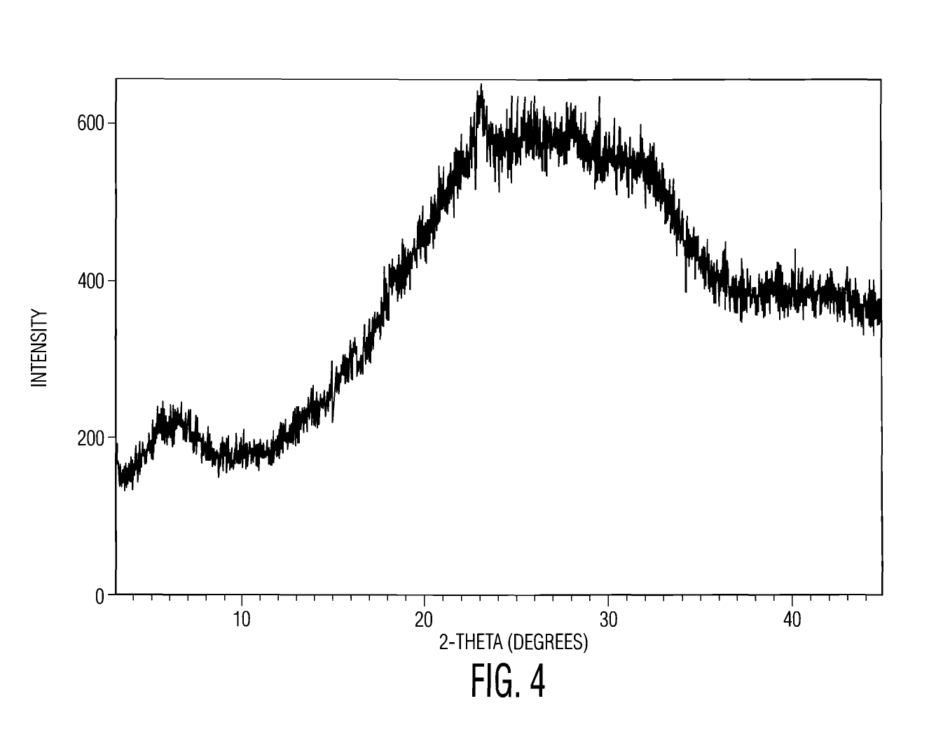

FIG. 5 is an illustration of an X-ray powder diffraction pattern of amorphous pemetrexed disodium, prepared according to Example 6.

FIG. 6 is an illustration of an X-ray powder diffraction pattern of amorphous pemetrexed disodium, after being stored for 22 days as in Example 6.

FIG. 7 is an illustration of an X-ray powder diffraction pattern of amorphous pemetrexed disodium, prepared according to Example 7.

FIG. 8 is an illustration of an X-ray powder diffraction pattern of pemetrexed disodium, prepared according to Example 1.

FIG. 9 is an illustration of an X-ray powder diffraction pattern of pemetrexed disodium heptahydrate, prepared according to Example 2.

### DETAILED DESCRIPTION

As set forth herein, aspects of the present application provide processes for the preparation of amorphous pemetrexed disodium, wherein the amorphous form may be characterized by its X-ray powder diffraction (XRPD) pattern, thermal techniques such as differential scanning calorimetry (DSC), Fourier-transform infrared (FT-IR) spectrometry, and/or thermogravimetric analysis (TGA). XRPD data reported herein were obtained using a Bruker AXS D8 Advance Powder X-ray Diffractometer with copper Kα radiation.

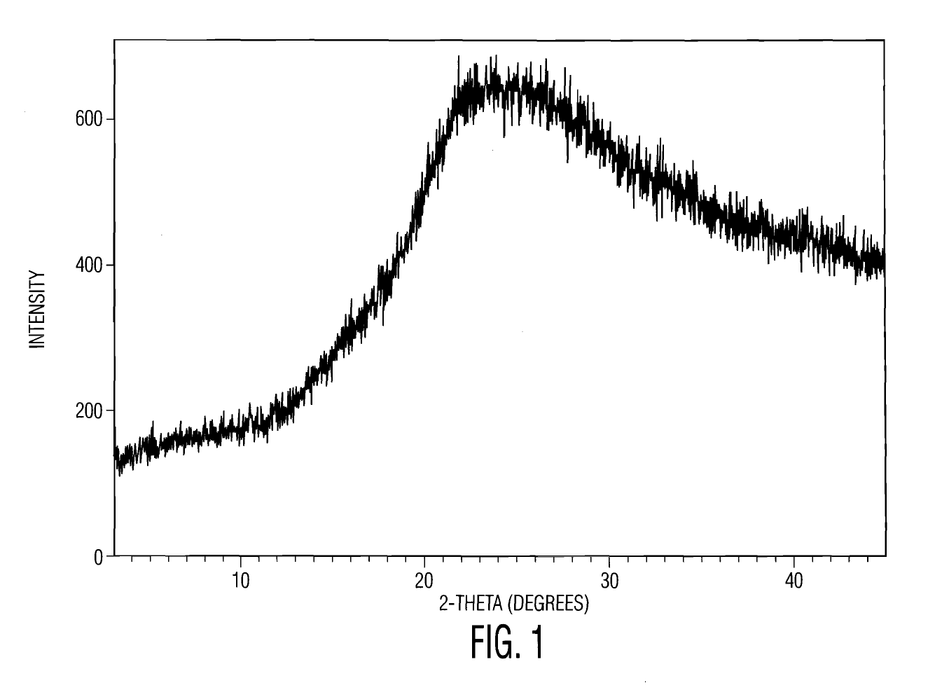

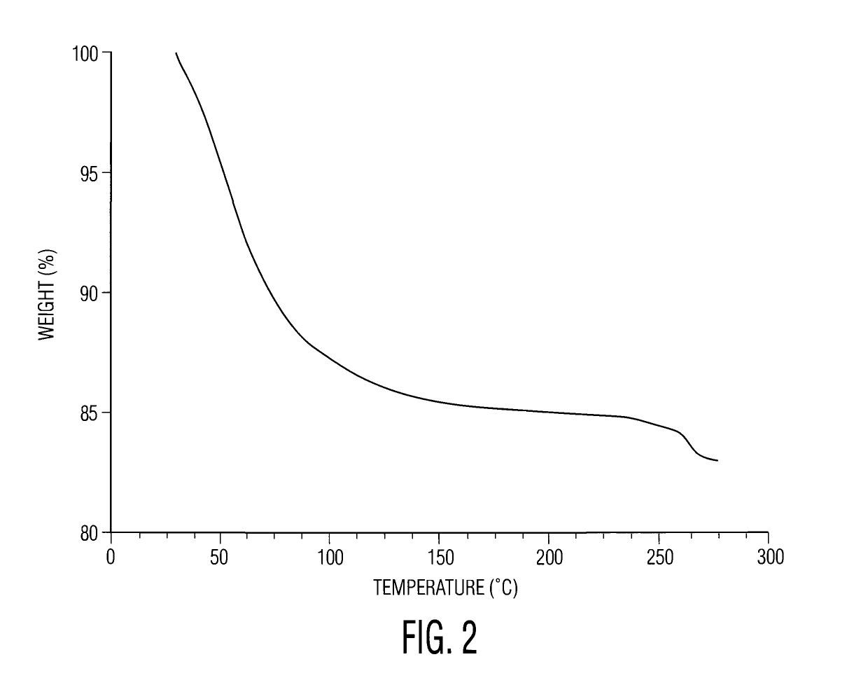

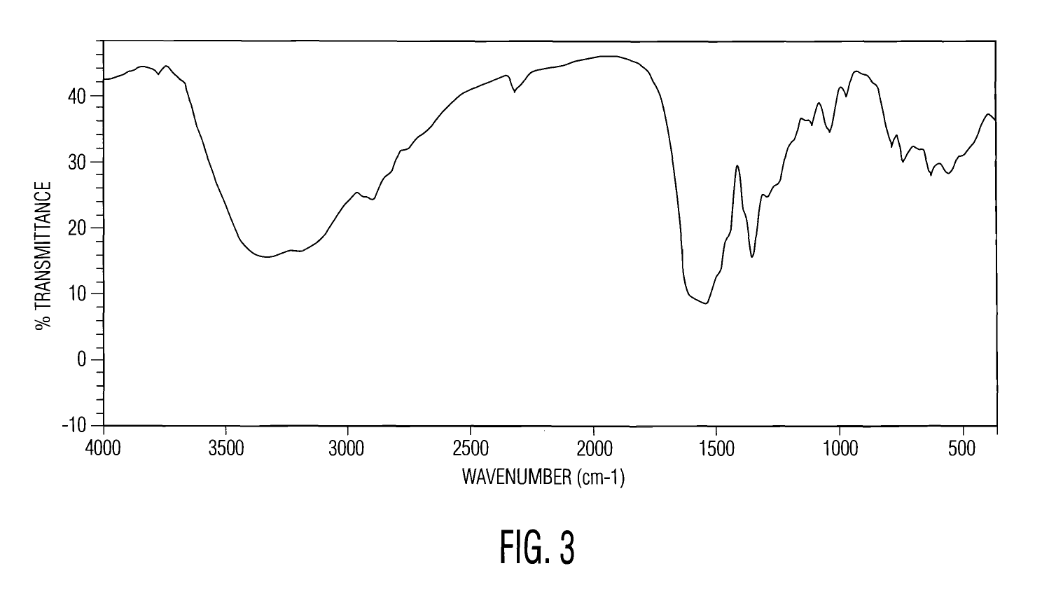

In an aspect, there are provided processes for preparing amorphous pemetrexed disodium by combining an anti-solvent with a solution of pemetrexed disodium. The amorphous pemetrexed disodium obtained from the present application can be characterized by any one or more of: its XRPD pattern, substantially in accordance with FIG. 1; its TGA curve, substantially in accordance with FIG. 2; and its infrared absorption spectrum in a potassium bromide (KBr) pellet, substantially in accordance with FIG. 3.

In accordance with an embodiment, a process for the preparation of amorphous pemetrexed disodium comprises:
a) providing a solution of pemetrexed disodium in a solvent;
b) precipitating a solid from the solution of a) by combining with an anti-solvent; and
c) recovering solid amorphous pemetrexed disodium.

Individual steps of the present process are discussed in detail herein below.

Step a) involves providing a solution of pemetrexed disodium. The solution of pemetrexed disodium may be obtained by dissolving pemetrexed disodium in a suitable solvent or a mixture of solvents, or such a solution may be obtained directly from a reaction in which pemetrexed disodium is formed. Any polymorphic form may be used in the preparation of a solution, such as crystalline forms, including solvates and hydrates. Examples of useful crystalline forms are the hemipentahydrate or heptahydrate of pemetrexed disodium. Solvents that may be used for dissolving pemetrexed disodium include, but are not limited to: C₁-C₄ alcohols, C₁-C₄ alkyl nitriles, C₃-C₅ alkyl amides, and any mixtures thereof. For example, the solvents that may be utilized include methanol, ethanol, acetonitrile, dimethylformamide, dimethylacetamide, water, and any mixtures thereof.

The dissolution temperatures can range from about 20° C. to about 100° C., depending on the solvent used for dissolution. Any other temperatures also are acceptable, as long as quality is not affected while a clear solution of pemetrexed disodium is provided. The quantity of solvent used for dissolution depends on the solvent and the dissolution temperature adopted. The concentration of pemetrexed disodium in the solution may generally range from about 0.02 g/mL to about 10 g/mL, however, other suitable concentrations may also be contemplated. In general, the use of higher concentrations, up to the saturation concentrations at elevated temperatures, will provide a more complete recovery of product. Optionally, the solution obtained above may be clarified to remove the undissolved particles followed by further processes. The undissolved particles may be removed suitably by filtration, centrifugation, decantation, and other techniques. The solution may be filtered by passing through paper, glass fiber, or other membrane material, or a bed of a clarifying agent such as Celite. Depending upon the equipment used and the concentration and temperature of the solution, the filtration apparatus may need to be heated to avoid premature precipitation.

Step b) involves precipitating the solid from solution of step a) by combining with an anti-solvent. The precipitation of solid may be performed by combining the solution of a) with an anti-solvent. The formation of amorphous solid may be initiated by adding the solution to the anti solvent, or adding anti solvent to the solution, at temperatures about 25° C. to about 35° C. Useful anti-solvents include ethers such as diethyl ether, dimethyl ether, di-isopropyl ether, methyl t-butyl ether, tetrahydrofuran, 1,4-dioxane, methyl isobutyl ketone, methyl ethyl ketone, and the like. The quantities of anti solvent used for solid precipitation may range from about 10 to 100 times, or about 60 to 80 times, the weight of pemetrexed disodium. Optionally, the suspension obtained may be cooled to a desired temperature and may then be stirred for a desired time, such as about 30 minutes to about 2 hours, or longer, depending upon the desired extent of precipitation. Solid precipitation may also be facilitated using methods such as cooling, partial removal of the solvent from the mixture, seeding, or a combination thereof. The solid precipitation may be performed using cooling and/or concentrating of the solution obtained from a).

The solution may be cooled and maintained further at temperatures lower than the dissolution temperatures, such as, for example, below about 10° C. to about 35° C., for a period of time as desired to precipitate the solid. The exact cooling temperatures and times required for complete precipitation can be readily determined by a person skilled in the art. Optionally, the solution may be subjected to concentration to an extent where the formation of amorphous pemetrexed disodium begins, or concentration performed to produce a weight ratio of solvent to pemetrexed disodium about 1:5 to about 1:20, to form a slurry. The slurry may be maintained further at temperatures lower than the concentration temperatures such as, for example, below about 10° C. to about 35° C., for a period of time as required for a more complete precipitation of the product. Concentration may be carried out suitably using evaporation, atmospheric distillation, or distillation under vacuum. Distillation of the solvent may be conducted under a vacuum of about 100 mm Hg to about 720 mm Hg, at temperatures of about 40° C. to about 70° C. Any temperature and vacuum conditions can be used as long as the concentration occurs without increases in the impurity levels.

Step c) involves recovering amorphous pemetrexed disodium. The solid can be recovered using any techniques such as filtration by gravity or by suction, centrifugation, decantation, and the like.

In an aspect, there are provided processes for preparing amorphous pemetrexed disodium, an embodiment of which comprises:

(a) providing a solution of pemetrexed disodium in an alcohol solvent;

(b) heating the solution of (a) to about 55-65° C.; and

(c) recovering amorphous pemetrexed disodium from step (b).

Individual steps of the present process are discussed in detail herein below.

Step (a) involves providing a solution of pemetrexed disodium in an alcohol solvent. The solution of pemetrexed disodium may be obtained by dissolving pemetrexed disodium in a suitable solvent or a mixture of solvents, or such a solution may be obtained directly from a reaction in which pemetrexed disodium is formed. Any polymorphic form may be used in the preparation of solution such as crystalline forms including solvates and hydrates. In embodiments, the crystalline form of pemetrexed disodium is the hemipentahydrate (2.5 hydrate) or heptahydrate of pemetrexed disodium. Alcohol solvents that may be used for dissolving pemetrexed disodium include, but are not limited to, C₁-C₄ alcohols, such as, for example, methanol, ethanol, isopropanol, n-butanol, and combinations thereof. Dissolution temperatures may range from about −5° C. to about 40° C. or higher. For example, the dissolution temperatures may be about 0-5° C. or about 0-10° C. The dissolution of pemetrexed disodium and the amount of solvent used for dissolution depend on temperatures at which dissolution takes place.

The concentration of pemetrexed disodium in the solution is not critical as long as sufficient solvent is employed to ensure total dissolution. For example, concentrations of pemetrexed disodium in a solvent may be less than or equal to about 25% by weight. The quantities of solvent used for dissolution are usually kept to a minimum, so as to avoid excessive product losses during product formation and isolation. In embodiments, the quantities of solvent may be greater than or equal to about 5 mL, per gram of pemetrexed disodium. Optionally, molecular sieves or any other suitable desiccants also may be added to the solution to control the moisture content.

A solution that is prepared may be filtered to remove any undissolved matter. The undissolved matter may be removed by filtration, centrifugation, decantation, and other techniques. The solution may be filtered by passing through paper, glass fiber, a pressure Nutsche filter (PNF), or other membrane material, or a bed of a clarifying agent such as diatomaceous earth. Optionally, the obtained filtrate may be concentrated to a desired level prior to the formation of amorphous product.

Step (b) involves heating the solution obtained from step (a) to about 55-65° C. The rate of heating required to get the temperature to about 55-65° C. may be readily determined by a person skilled in the art. For example, the rate of heating required to get the temperature may be about 0.5° C./minute to about 3° C./minute.

Optionally, the solution may be filtered at temperatures about 30-35° C. while heating to about 55-65° C., to improve the color of amorphous pemetrexed disodium, for example, to remove a greenish tinge. The solution may be held at about 55-65° C., prior to precipitation of the amorphous compound. Initiation of precipitation of amorphous compound can begin during reflux or while heating the reaction solution to reflux. The reaction mass may be held at about 55-60° C. for a sufficient period of time, for example from about 10 to about 45 minutes. The formation of the amorphous solid may be enhanced by using any suitable method, such as cooling, slurrying, and/or combining with an anti-solvent. The exact cooling temperature and time required for complete isolation can be readily determined by a person skilled in the art. For example, the rate of cooling may range from about 0.5 to about 3° C./minute. This, however, depend on the reaction vessel, volume of the vessel, stirrer, and the like. The mass may be stirred below 35° C. for a period of about 30 minutes or more. Suitable anti-solvents include, but are not limited to, ethers, for example, diisopropyl ether, diethyl ether, methyl t-butyl ether, and the like.

Step (c) involves recovering solid amorphous pemetrexed disodium. The formed solid may be recovered, for example, using filtration by gravity or suction, centrifugation, decantation, and the like. The obtained amorphous pemetrexed disodium from the present process is stable during storage for more than about 20 days at room temperature, as is shown in Example 6.

A specific embodiment of the present invention provides a process for preparing amorphous pemetrexed disodium, comprising:

(a) providing a solution of pemetrexed disodium in methanol below about 35° C.;

(b) heating the solution to about 55-65° C.;

(c) cooling the solution; and

(d) recovering amorphous solid pemetrexed disodium.

A specific embodiment of the present invention provides a process for preparing amorphous pemetrexed disodium, comprising:

(a) providing a solution of pemetrexed disodium in methanol below about 35° C.;

(b) heating the solution of (a) to about 55-65° C.;

(c) adding an anti-solvent to the solution of (b); and

(d) recovering amorphous solid pemetrexed sodium.

A specific embodiment of the present invention provides a process for preparing amorphous pemetrexed disodium, comprising:

(a) providing a solution of pemetrexed disodium in methanol below about 15° C.;

(b) heating the solution to about 30-35° C.;

(c) filtering the solution;

(d) heating the filtrate to reflux;

(e) cooling the filtrate; and

(d) recovering amorphous solid pemetrexed sodium.

The amorphous solid obtained from the present invention may be dried under suitable conditions to afford the desired pemetrexed disodium in a pure amorphous form, substantially free of residual solvents. Drying may be carried out under reduced pressure conditions until the residual solvent content reduces to an amount that is within the limits given by the International Conference on Harmonization of Technical Requirements for Registration of Pharmaceuticals for Human Use (“ICH”) guidelines. The guideline solvent level depends on the type of solvent, but is not more than about 5000 ppm, or about 4000 ppm, or about 3000 ppm. The drying may be carried out at reduced pressures, such as below about 680 mm Hg or below about 50 mm Hg, at temperatures such as about 20° C. to about 50° C. by using suitable equipments.

D. L. Teagarden, “Practical aspects of lyophilization using non-aqueous co-solvent systems,” European Journal of Pharmaceutical Sciences, 15 (2), (2002) pp. 115-133 generally discloses control of residual solvent levels by using humidification for amorphous carbohydrate systems. The process involves humidification of the dried carbohydrate containing the entrapped volatile organics, wherein there appears to be a critical humidity condition which results in a corresponding moisture level where the volatile organic is rapidly released. This article also discloses the removal of different residual solvents including acetone, ethanol, and methanol from the amorphous carbohydrate using humidification.

The present application further provides drying processes for amorphous pemetrexed disodium, using humidification with certain relative humidity (RH) conditions, such as below 75% RH, at temperatures such as about 20° C. to about 40° C. The drying may be performed for any desired time periods that achieve the desired product specifications, such as times from about 1 hour to about 20 hours, or longer. Drying may also be carried out for shorter or longer periods of time depending on the product specifications. Subsequent drying may be required to remove the water content suitably using equipment such as a pressure Nutsche filter (PNF), vacuum tray dryer (VTD), vacuum oven, air tray dryer (ATD), or using a fluidized bed dryer, spin flash dryer, flash dryer, humidification chamber or combinations thereof.

In an embodiment, there is provided a drying process for pemetrexed disodium, using equipment such as a humidification chamber, ATD and VTD, alone or in combination, to reduce the organic volatile impurities, followed by drying to a desired moisture content to meet the ICH guidelines. Drying by using VTD or ATD may be carried out at a temperature of about 20° C. to about 50° C.

In an embodiment, there is provided a process for preparing crystalline pemetrexed disodium heptahydrate, comprising:
a) providing a solution of pemetrexed disodium in water with inert gas bubbling through the solution;
b) precipitating a solid from the solution of (a) by combining with an anti-solvent under inert gas bubbling;
c) isolating a solid from (b) under an inert atmosphere; and
d) drying the solid of (c) under vacuum to afford pemetrexed disodium heptahydrate.

The solution of pemetrexed disodium may be obtained by dissolving pemetrexed disodium in water, or a solution may be obtained directly from a reaction in which pemetrexed disodium is formed. Any polymorphic form may be used in the preparation of a solution, such as a crystalline form or amorphous material. Any temperatures may be used for dissolution as long as quality is not affected. The solution is provided with inert gas bubbling, such as using nitrogen.

Solid pemetrexed disodium is precipitated by combining the solution with an anti solvent. Suitable anti solvents include: ketones, such as acetone, methyl ethyl ketone, and the like; and alcohols, such as methanol, ethanol, isopropyl alcohol, butanol, and the like. The mode of addition may include adding a solution of pemetrexed disodium into an anti-solvent, or vice versa. The precipitated solid is isolated using any techniques such as filtration by gravity or by suction, centrifugation, decantation, and the like, in an inert atmosphere, such as a nitrogen atmosphere. Drying may be carried out under reduced pressures, such as below about 680 mm Hg or below about 50 mm Hg, at temperatures such as about 20° C. to about 40° C., to obtain the crystalline pemetrexed disodium heptahydrate.

### DEFINITIONS

All percentages and ratios used herein are by weight of the total composition and all measurements made are at 25° C. and atmospheric pressure unless otherwise designated. All temperatures are in degrees Celsius unless specified otherwise. The present invention can comprise (open ended) or consist essentially of the components of the present invention as well as other ingredients or elements described herein. As used herein, “comprising” means the elements recited, or their equivalent in structure or function, plus any other element or elements which are not recited. The terms “having” and “including” are also to be construed as open ended unless the context suggests otherwise. As used herein, “consisting essentially of means that the invention may include ingredients in addition to those recited in the claim, but only if the additional ingredients do not materially alter the basic and novel characteristics of the claimed invention. In embodiments, such additives will not be present at all or only in trace amounts. However, it may be possible to include up to about 10% by weight of materials that could materially alter the basic and novel characteristics of the invention as long as the utility of the compounds (as opposed to the degree of utility) is maintained. All ranges recited herein include the endpoints, including those that recite a range “between” two values. Terms such as “about,” “generally,” “substantially,” and the like are to be construed as modifying a term or value such that it is not an absolute, but does not read on the prior art. Such terms will be defined by the circumstances and the terms that they modify as those terms are understood by those of skill in the art. This includes, at very least, the degree of expected experimental error, technique error and instrument error for a given technique used to measure a value.

Note that while the specification and claims may refer to a final product such as, for example, a tablet or other dosage form of the invention as, for example, containing particles having a certain particle size or distribution, or a certain type of, for example, a specific form of a filler, it may be difficult to tell from the final dosage form that the recitation is satisfied. However, such a recitation may be satisfied if the materials used prior to final production (in the case of a tablet for example, blending and tablet compression), for example, meet that recitation. Indeed, as to any property or characteristic of a final product which cannot be ascertained from the dosage form directly, it is sufficient if that property resides in the components recited just prior to final production steps.

Where this document refers to a material, such as in this instance, pemetrexed, and the solid forms, salts, solvates and/or optical isomers thereof by reference to patterns, spectra or other graphical data, it may do so by qualifying that they are “substantially” shown or depicted in a Figure, or by one or more data points. By “substantially” used in such a context, it will be appreciated that patterns, spectra and other graphical data can be shifted in their positions, relative intensities, or other values due to a number of factors known to those of skill in the art. For example, in the crystallographic and powder X-ray diffraction arts, shifts in peak positions or the relative intensities of one or more peaks of a pattern can occur because of, without limitation: the equipment used, the sample preparation protocol, preferred packing and orientations, the radiation source, operator error, method and length of data collection, and the like. However, those of ordinary skill in the art should be able to compare the figures herein with a pattern generated of an unknown form of, in this case, pemetrexed, and confirm its identity as one of the forms disclosed and claimed herein. The same holds true for other techniques which may be reported herein. This is true for amorphous solids as well, although there are not specific peaks.

In addition, where a reference is made to a drawing figure, it is permissible to, and this document includes and contemplates, the selection of any number of data points illustrated in the figure which uniquely define that form, salt, solvate, and/or optical isomer, within any associated and recited margin of error, for purposes of identification.

A reference to a molecule such as, in this case, pemetrexed, unless otherwise specified or inconsistent with the disclosure in general, refers to the base compound, and any salt, amorphous form, optical isomer, and/or solvate forms thereof.

When a molecule or other material is identified herein as “pure”, it generally means, unless specified otherwise, that the material is about 99% pure or higher. In general, this refers to purity with regard to unwanted residual solvents, reaction by-products, impurities, and unreacted starting materials. In the case of polymorphs or other solid forms such as crystals, “pure” can also mean 99% of the amorphous form with regard to crystalline forms, as appropriate. “Substantially” pure means, the same as “pure” except that the lower limit is about 98% pure or higher, and, likewise, “essentially” pure means the same as “pure” except that the lower limit is about 95% pure.

Certain specific aspects and embodiments of the present application will be explained in more detail with reference to the following examples, which are provided only for purposes of illustration and should not be construed as limiting the scope of the invention in any manner.

### EXAMPLES

### Example 1

### Preparation of Pemetrexed Disodium

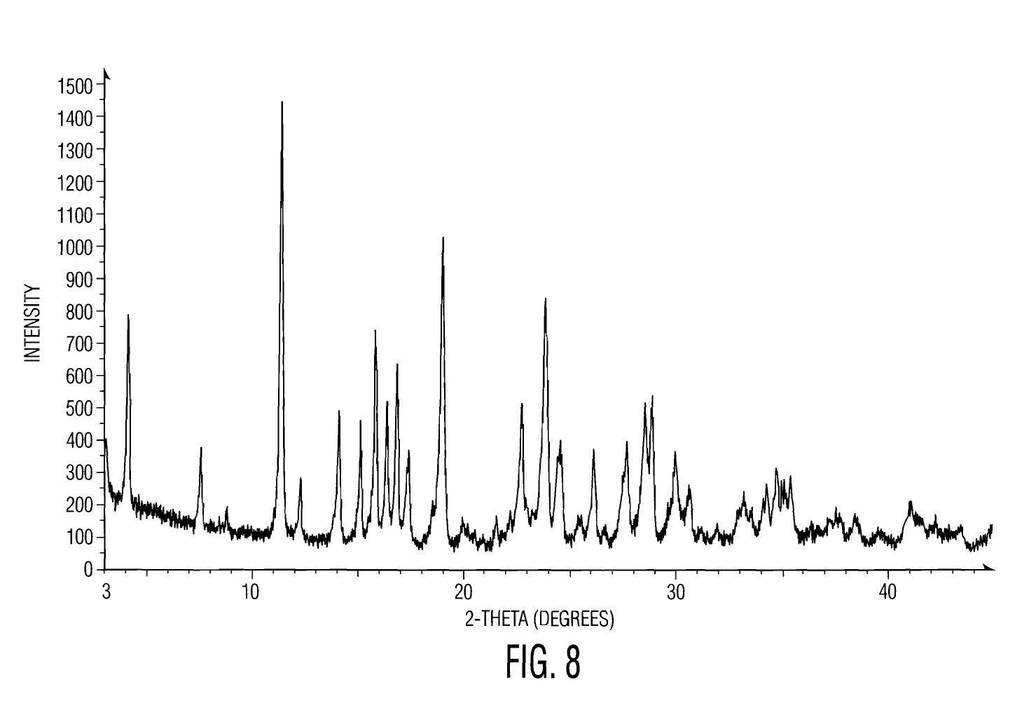

N-[4-[2-(2-Amino-4,7-dihydro-4-oxo-1H-pyrrolo[2,3-d]pyrimidin-5-yl)ethyl]benzoyl]-glutamic acid dimethyl ester p-toluenesulfonate salt (50 g) is dissolved in sodium hydroxide solution (19.1 g of sodium hydroxide dissolved in 478 mL water) at 0-5° C. under nitrogen and stirred for 45 minutes. The reaction solution is filtered and the filtrate is adjusted to pH 7.79 by adding 1N HCl solution (190 mL). The mixture is added to acetone (1500 mL) under nitrogen, stirred for 1 hour at 25-30° C., and filtered. The solid is washed with acetone (150 mL) and dried at 30-35° C. under vacuum for 2 hours, to afford 43 g of the title compound. The XRPD pattern is substantially as shown in FIG. 8. Moisture content: 21.76% by the Karl Fisher method. Purity: 99.58% by HPLC; 0.32% p-toluenesulfonic acid; 0.03% compound at 0.90 RRT. Chiral purity: 99.95% by chiral HPLC.

### Example 2

### Preparation of Pemetrexed Disodium Heptahydrate

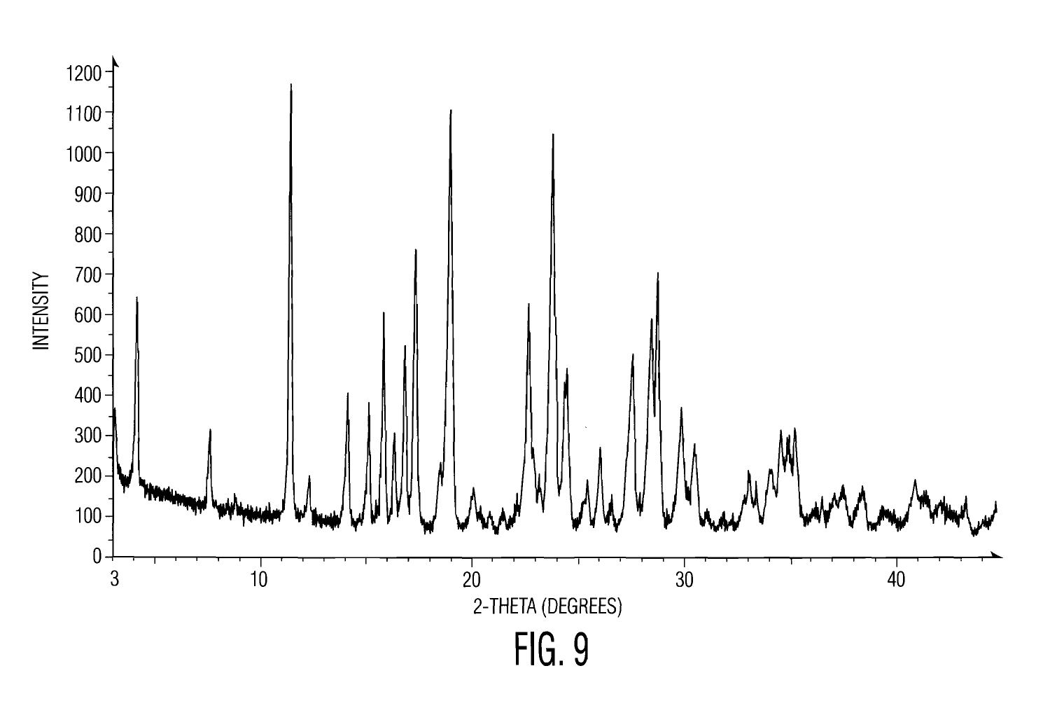

Pemetrexed disodium (30 g) obtained according to Example 1 is dissolved in water (210 mL) with nitrogen gas bubbling. The solution is filtered and the filtrate is added to acetone (1.2 L) over 30-45 minutes with nitrogen bubbling through the solution. The mixture is stirred for 60 minutes at 25-30° C. with nitrogen bubbling, filtered, and the solid is washed with acetone (90 mL) under a nitrogen atmosphere. The solid is dried at 30-35° C. under high vacuum (not less than 680 mm Hg) for about 4 hours to afford 26.8 g of title compound. The XRPD pattern is substantially as shown in FIG. 9. Purity: 99.88% by HPLC; 0.03% compound at 0.89 RRT, 0.02% compound at 0.90 RRT, 0.01% compound at 0.91 RRT, 0.01% p-toluenesulfonic acid. Chiral purity: 99.96%. Moisture content: 22.05% by the Karl Fisher method.

### Example 3

### Preparation of Amorphous Pemetrexed Disodium

Pemetrexed disodium (1 g) is dissolved in methanol (25 mL) at 25 to 30° C. and the whole solution is filtered to remove any undissolved particles. Diisopropyl ether (70 mL) is added to the filtrate at the same temperature and stirred for 1 hour. The suspension is filtered and the solid is washed with diisopropyl ether (10 mL) and then dried under vacuum at 45 to 50° C. for 2 hours, to obtain 0.84 g of pemetrexed disodium in amorphous form. Moisture content (MC): 15.69% w/w by the Karl Fisher method.

### Example 4

### Preparation of Amorphous Pemetrexed Disodium

Pemetrexed disodium (1 g) is dissolved in methanol (30 mL) at 25 to 30° C. and filtered to remove any undissolved particles. T-butyl methyl ether (70 mL) is added to the filtrate at the same temperature and stirred for 1 hour. The suspension is filtered and the solid is washed with t-butyl methyl ether (10 mL) and dried under vacuum at 45 to 50° C. for 4 hours, to obtain 0.5 g of amorphous pemetrexed disodium. MC: 9.15% w/w by the Karl Fisher method.

### Example 5

### Preparation of Amorphous Pemetrexed Disodium

Pemetrexed disodium (1 g) is dissolved in methanol (30 mL) at 25 to 30° C., then the solution is cooled to 0° C. and stirred for 3 hours. The solvent is distilled at about 45° C. under vacuum until 10 to 15 mL remained. The mass is cooled to 0° C. and stirred for 1 hour. The suspension is filtered and the solid is dried at 40-45° C. for 4 hours, to afford 0.39 g of title compound.

### Example 6

### Preparation of Amorphous Pemetrexed Disodium

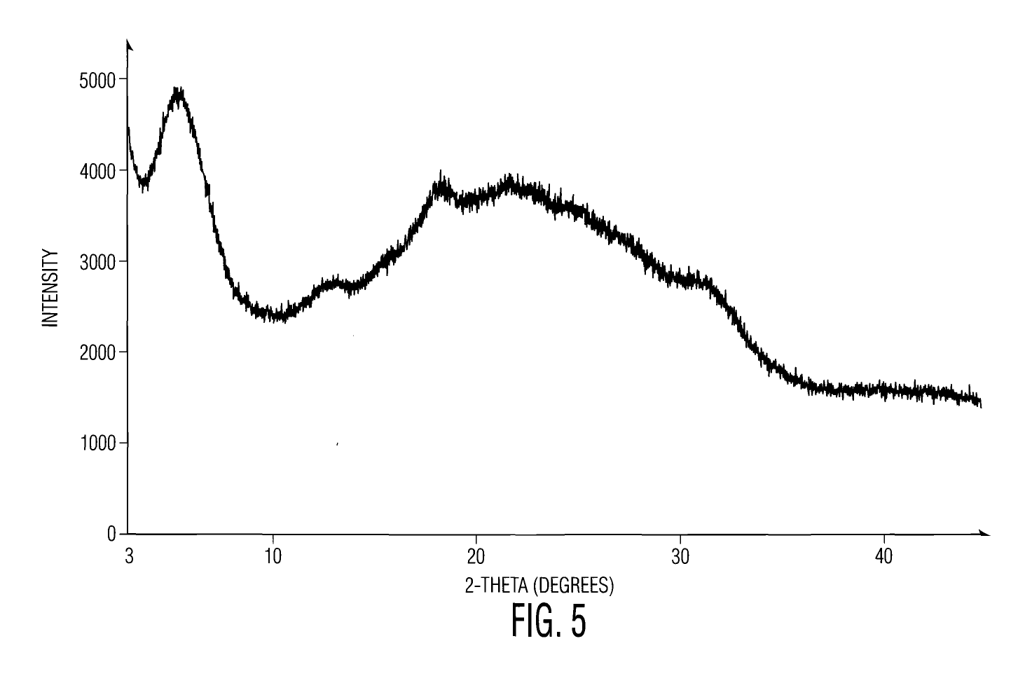

Pemetrexed disodium heptahydrate (40 g) is dissolved in methanol (400 mL) at 0-5° C. The solution is filtered and the filtrate is heated to reflux over 15-20 minutes, and cooled to 0-5° C. over 30 minutes. The mass is stirred for 45-60 minutes at 0-5° C. The formed suspension is filtered and the solid is dried in a vacuum tray drier at 25-35° C. for 2-3 hours, to afford 16.5 g of amorphous pemetrexed disodium. Yield: 42%. The XRPD pattern is substantially as shown in FIG. 5.

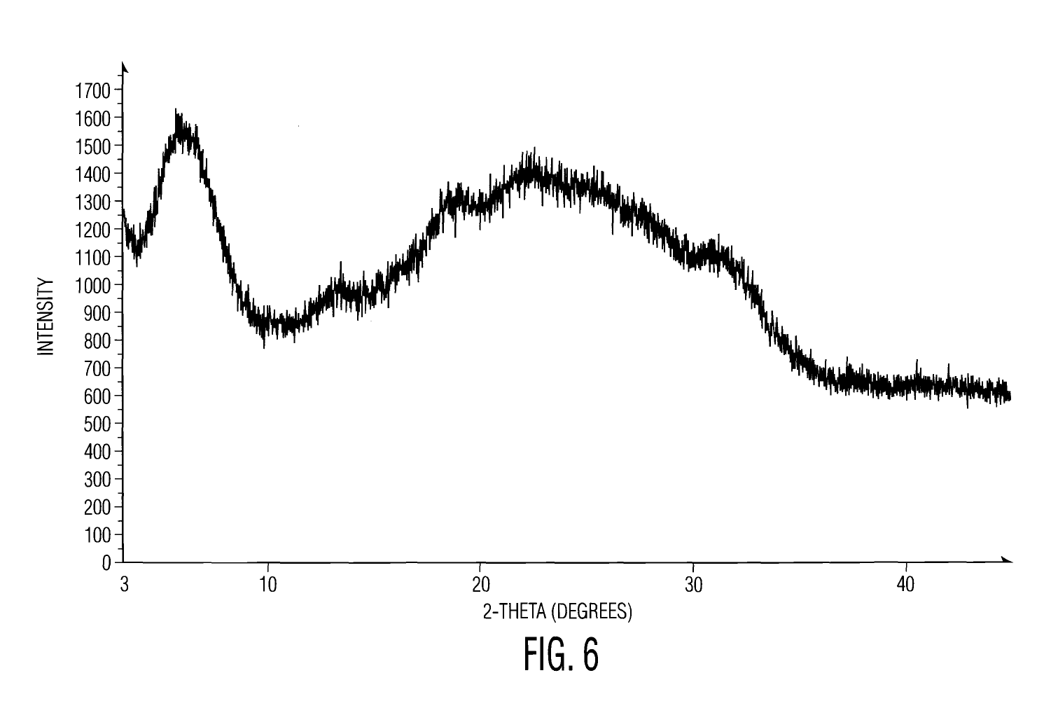

A sample of the product is packaged in a sealed polyethylene bag, placed inside a sealed triple laminated bag. The package is stored at temperatures of about 25-35° C. for 22 days, and the stored material is analyzed by X-ray diffraction. The sample is found to retain its polymorphic form after 22 days, as shown by comparing the original XRPD pattern as substantially shown in FIG. 5 to the XRPD pattern of the stored sample as substantially shown in FIG. 6.

### Example 7

### Preparation of Amorphous Pemetrexed Disodium

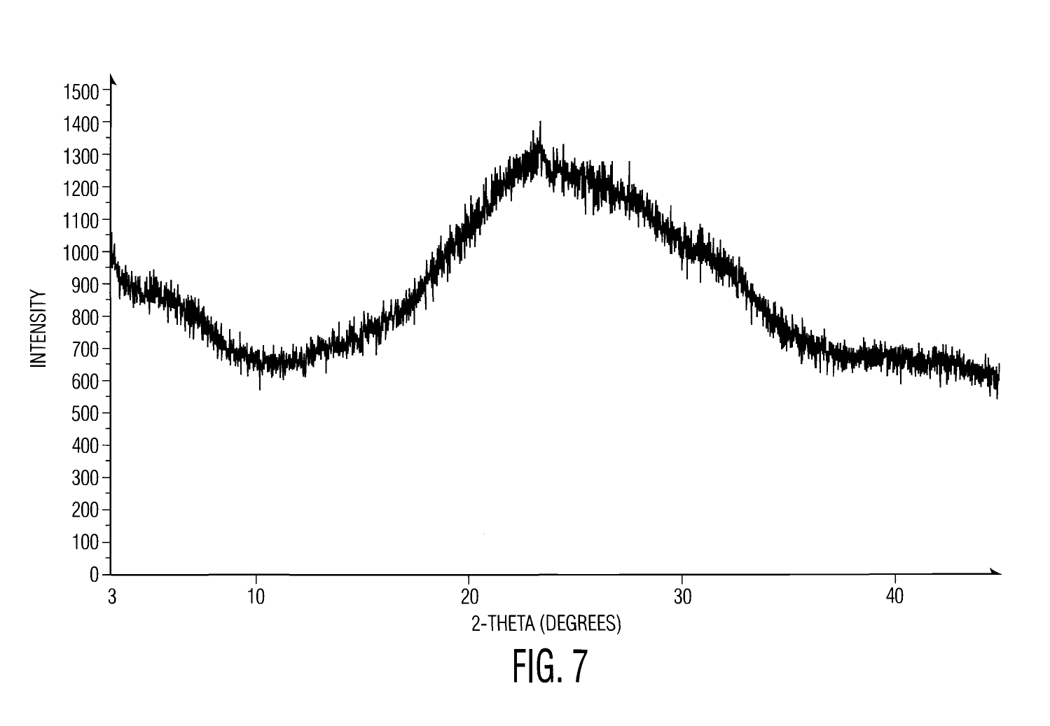

Pemetrexed disodium 2.5 hydrate (2 g) is dissolved in methanol (50 mL) at 0-5° C. The solution is filtered and the filtrate is heated to reflux over 15-20 minutes, cooled to 25-35° C. over 20 minutes, and stirred for 1 hour at that temperature. The formed suspension is filtered and the solid is dried in a vacuum tray dryer at 25-35° C. for 2-3 hours, to afford 0.9 g of amorphous pemetrexed disodium. Yield: 45%. The XRPD pattern is substantially as shown in FIG. 7.

### Example 8

### Preparation of Amorphous Pemetrexed Disodium

Pemetrexed disodium (10 g) is dissolved in methanol (100 mL) at 0-5° C. The solution is heated to 30-35° C. and filtered to remove unwanted solids with vacuum. The filtrate is heated to reflux over 30 minutes, stirred for 10 minutes, cooled to 25-35° C. over 60 minutes, and stirred for 15 minutes at that temperature. The formed suspension is filtered and the solid is dried in a pressure Nutsche filter (PNF) for 10-15 minutes. The solid is dried in a vacuum tray dryer at 25-35° C. for 3 hours under vacuum, and the solid is further dried in a humidification chamber for 6 hours at 25-30° C. and 60% RH. Finally, the solid is dried in a vacuum tray dryer at 25-35° C. for 5 hours, to afford 3.8 g of amorphous pemetrexed disodium. XRPD pattern shows amorphous character. Residual methanol: 164 ppm by gas chromatography. MC: 13.44% w/w by the Karl Fisher method. Purity by HPLC: 99.55%. Purity by chiral HPLC: 99.91%.

### Example 9

### Preparation of Amorphous Pemetrexed Disodium

Pemetrexed disodium heptahydrate (4 g) is dissolved in methanol (40 mL) at 0-5° C. The solution is filtered and the filtrate is heated to reflux over 15-20 minutes, cooled to 25-35° C. over 15-20 minutes, and stirred at same temperature for 45 minutes. The formed suspension is filtered and the solid is dried in a vacuum tray dryer at 25-35° C. for 2 hours, to afford 1.8 g of amorphous pemetrexed disodium. Yield: 50%. MC: 10.705% w/w by the Karl Fisher method.

### Example 10

### Preparation of Amorphous Pemetrexed Disodium

Pemetrexed disodium heptahydrate (1 g) is dissolved in methanol (10 mL) at 0-5° C. The solution is filtered and the filtrate is heated to reflux over 10 minutes and cooled to 25-35° C. over 15-20 minutes. Diisopropyl ether (20 mL) is added and stirred for 1 hour. The suspension is filtered and the solid is dried in a vacuum tray dryer at 25-35° C. for 2-3 hours, to afford 760 mg of amorphous pemetrexed disodium. Yield: 76%.

### Example 11

### Preparation of Amorphous Pemetrexed Disodium

Pemetrexed disodium heptahydrate (1 g) is dissolved in methanol (10 mL) at 0-5° C. The solution is filtered and the filtrate is heated to reflux and cooled to 25-35° C. in 15-20 minutes. Methyl t-butyl ether (100 mL) is added over 15-30 minutes and stirred for 45-60 minutes. The formed suspension is filtered and the solid is dried in a vacuum tray dryer at 25-35° C. for 2-3 hours, to afford 880 mg of amorphous pemetrexed disodium. Yield: 88%.

### Example 12

### Preparation of Amorphous Pemetrexed Disodium

Pemetrexed disodium heptahydrate (2 g) is dissolved in methanol (10 mL) at 0-5° C. and molecular sieves (4 g) are added. The mass is filtered and the filtrate is heated to reflux, cooled to 25-35° C. over 15-20 minutes, and stirred for 45-60 minutes. The formed suspension is filtered and the solid is dried in a vacuum tray dryer at 25-35° C. for 2-3 hours, to afford 900 mg of amorphous pemetrexed disodium.

### Example 13

### Removal of Organic Volatile Impurities/Residual Solvents from Amorphous Pemetrexed Disodium

Pemetrexed disodium amorphous (9 g; methanol content: 15,234 ppm; moisture content: 4.25% w/w by KF) is maintained under humidification at 60% RH, at 25° C., for 4.5 hours and then a sample is analyzed for residual solvent content. Methanol: 2,285 ppm. Moisture content: 12.35% w/w by the Karl Fischer method.

The remaining material is further exposed to humidification at 60% RH, at 25° C. for 2.5 hours and a sample is analyzed for residual solvent content. Methanol: 583 ppm. Moisture content: 12.9% w/w by the Karl Fischer method.

The remaining material is dried in a vacuum tray dryer at 30° C. for 5 hours, and is found to have a moisture content of 6.44% w/w by KF.

### Example 14

### Preparation of Amorphous Pemetrexed Disodium

Pemetrexed disodium heptahydrate (40 g) is dissolved in methanol (400 mL) at 0-5° C. The solution is filtered and the filtrate is heated to reflux over 15-20 minutes, and then cooled to 25-35° C. over 20 minutes. The mass is stirred for 15-20 minutes at 25-35° C. The formed suspension is filtered with a pressure Nutsche filter, to obtain 29 g of wet amorphous pemetrexed disodium.

A portion of wet pemetrexed disodium (13.5 g) is washed with n-butyl acetate (3×13 mL) and the solid is dried in a vacuum tray dryer at 40° C. for 11 hours, to afford 9.1 g of Sample 1.

### Sample 1 Analyses

| | |
|---|---|
| XRPD | Amorphous |
| MC | 5.52% w/w |
| Residual solvents | Methanol: 2,787 ppm |
| | n-Butyl acetate: 584 ppm |

### Example 15

### Preparation of Amorphous Pemetrexed Disodium

Pemetrexed disodium heptahydrate (30 g) is dissolved in methanol (300 mL) at 0-5° C. The solution is filtered and the filtrate is heated to reflux over 15-20 minutes, then cooled to 25-35° C. over 20 minutes. The mass is stirred for 15-20 minutes at 25-35° C. The formed suspension is filtered using a PNF under a nitrogen atmosphere and the solid is dried in a vacuum tray dryer at 40° C. for 10-11 hours, to afford 14.8 g of amorphous pemetrexed disodium. Dried pemetrexed disodium (5.5 g) is placed into a Petri dish and dried in air (in an ATD) at 40° C. for 20 hours, to obtain 5.71 g of Sample 1.

Pemetrexed disodium (9.3 g) obtained from VTD drying is micronized at 25° C. using 3 kg/cm² pressure. The micronized solid is air dried (in an ATD) for 10-12 hours at 40° C., to obtain 7.0 g of Sample 2.

| Analysis | Sample 1 | Sample 2 |
|---|---|---|
| XRPD | Amorphous | Amorphous |
| MC | 7.10% w/w | 7.21% w/w |
| Methanol | 5112 ppm | 882 ppm |

Throughout this application, various publications are referenced. The disclosures of these publications in their entireties are hereby incorporated by reference into this application in order to more fully describe the state of the art as known to those skilled therein as of the date of the application described and claimed herein.

While particular embodiments of the present application have been illustrated and described, it would be obvious to those skilled in the art that various other changes and modifications can be made without departing from the spirit and scope of the application. It is therefore intended to cover in the appended claims all such changes and modifications that are within the scope of this invention.

What is claimed is:

1. A process for preparing amorphous pemetrexed disodium, comprising:
(a) providing a solution of pemetrexed disodium in a solvent or a mixture of solvents;
(b) precipitating a solid from the solution of (a) by combining with an anti-solvent; and
(c) recovering solid amorphous pemetrexed disodium.

2. The process of claim 1, wherein the solution of pemetrexed disodium in (a) is prepared using a crystalline hydrate.

3. The process of claim 1, wherein a solvent comprises a C₁-C₄ alcohol, C₁-C₄ alkyl nitrile, C₃-C₅ alkyl amide, ketone, or a mixture of two or more thereof.

4. The process of claim 1, wherein an anti-solvent comprises an ether.

5. A process for preparing amorphous pemetrexed disodium, comprising:
(a) providing a solution of pemetrexed disodium in an alcohol solvent;
(b) heating the solution to about 55-65° C.; and
(c) recovering amorphous pemetrexed disodium.

6. The process of claim 5, wherein an alcohol comprises methanol.

7. The process of claim 5, wherein a solution in (a) is formed at about 0° C. to about 10° C.

8. The process of claim 5, wherein heating in (b) is at rates about 0.5 to about 3° C./minute.

9. The process of claim 5, further comprising combining a solution with an anti-solvent at temperatures about 55-65° C., following (b).

10. The process of claim 9, wherein an anti-solvent comprises a ketone.

11. The process of claim 5, further comprising cooling a solution, following (b).

12. The process of claim 11, wherein cooling is to temperatures below about 30° C.

13. A process for preparing amorphous pemetrexed disodium, comprising:
(a) providing a solution of pemetrexed disodium in methanol below 35° C.;
(b) heating the solution to about 55-65° C.;
(c) cooling the solution; and
(d) recovering amorphous pemetrexed disodium.

14. A process for preparing amorphous pemetrexed disodium, comprising:
(a) providing a solution of pemetrexed disodium in methanol below 35° C.;
(b) heating the solution to about 55-65° C.;
(c) combining the solution with an anti-solvent; and
(d) recovering amorphous pemetrexed disodium.

15. A process for removing residual solvents from amorphous pemetrexed disodium, comprising:
(a) exposing amorphous pemetrexed disodium to relative humidity below about 75 percent at temperatures about 20° C. to about 40° C., for a time that reduces the residual solvent content; and
(b) optionally, drying exposed amorphous pemetrexed disodium at temperatures about 20° C. to about 50° C.

16. A process for preparing crystalline pemetrexed disodium heptahydrate, comprising:
(a) providing a solution of pemetrexed disodium in water with inert gas bubbling;
(b) precipitating a solid from the solution of (a) by combining with an anti-solvent with inert gas bubbling;
(c) isolating the solid from (b) under an inert gas atmosphere; and
(d) drying the solid of (c) under vacuum to afford crystalline pemetrexed disodium heptahydrate.

17. The process of claim 16, wherein an anti-solvent comprises a ketone or an alcohol.

18. The process of claim 16, wherein drying is conducted under vacuum below about 680 mm Hg.

19. The process of claim 16, where an inert gas comprises nitrogen.
